(19) United States
(12) Patent Application Publication
(10) Pub. No.: US 2026/0364037 A1
(43) Pub. Date: Nov. 19, 2026

(54) COMPOSITION CONTAINING GAMMA-AMINOBUTYRIC ACID

(71) Applicants: Pharma Foods International Co., Ltd., Kyoto (JP); Nakamura Sangyo Gakuen, Fukuoka (JP)

(72) Inventors: Yoshihiro Hara, Kyoto (JP); Utano Nakamura, Kyoto (JP); Keita Koga, Kyoto (JP); Riho Sagane, Kyoto (JP); Yusuke Yamashita, Kyoto (JP); Mujo Kim, Kyoto (JP); Goichi Hagiwara, Fukuoka (JP); Ryousuke Furukado, Fukuoka (JP)

(73) Assignees: Pharma Foods International Co., Ltd., Kyoto (JP); Nakamura Sangyo Gakuen, Fukuoka (JP)

(21) Appl. No.: 19/515,385

(22) PCT Filed: Aug. 30, 2024
(86) PCT No.: PCT/JP2024/031209

§ 371 Date: Feb. 27, 2026

(30) Foreign Application Priority Data
Aug. 31, 2023 (JP) 2023-141354

(51) Int. Cl. A61K 31/197; A61P 25/00; A61P 25/28

(52) U.S. Cl. CPC A61K 31/197; A61P 25/00; A61P 25/28

(57) ABSTRACT

The present invention provides a composition for inducing a flow (zone) state in a subject, the composition containing γ-aminobutyric acid (GABA). The composition according to the present invention is capable of inducing a subject into a flow (zone) state upon carrying out a specific work or movement.

### TECHNICAL FIELD

The present invention is directed to a composition containing γ-aminobutyric acid (GABA), in particular, directed to a composition containing GABA for inducing a flow (zone) state.

### BACKGROUND ART

γ-aminobutyric acid (GABA, 4-aminobutyric acid) has been recently receiving attention as an amino acid that is widely distributed in the natural world and can be added to food without harming its taste. GABA is known to be a suppressive neurotransmitter, many of which exist in a central nervous system of a mammal, suppressing excessive secretion of excitatory neurotransmitter to relieve excitation of a nerve, thus exerting a relaxing effect or anti-stress action. In addition, it is known to have various physiological activities such as blood pressure lowering action, cholesterol reducing action and suppression of reduction of immunity. This GABA is also comprised in vegetables, grain and human bodies, and thus is easily added to food. Chocolates and many supplements containing GABA are sold.

### SUMMARY OF INVENTION

### Solution to Problem

The inventors of the present invention found out that GABA can induce a flow (zone) state in a subject.

Therefore, according to the major viewpoints of the present invention, the following inventions are provided.

### Item 1

A composition for inducing a flow (zone) state in a subject, comprising γ-aminobutyric acid (GABA).

### Item 2

The composition of the item above, which is characterized by being ingested before performing an assignment.

### Item 3

The composition of any one of the items above, wherein the flow (zone) state is defined at least by distortion of perceived time.

### Item 4

The composition of any one of the items above, wherein the flow (zone) state comprises shortening of perceived time.

### Item 5

The composition of any one of the items above, wherein the assignment comprises multiple simultaneous tasks.

### Item 6

The composition of any one of the items above, wherein the assignment comprises an e-sport, a VDT work, a digital work, and a game.

### Item 7

The composition of any one of the items above, wherein the game comprises a TV game, a PC game, and a mobile terminal game.

### Item 8

The composition of any one of the items above, which is ingested from at least about 2 hours before to immediately before performing the assignment.

### Item 9

The composition of any one of the items above, wherein a content amount of the γ-aminobutyric acid is about 10 to about 1000 mg.

### Item 10

The composition of any one of the items above, wherein an ingestion amount of the γ-aminobutyric acid is about 10 to about 1000 mg per day.

### Item 11

The composition of any one of the items above, which is ingested orally.

### Item 12

The composition of any one of the items above, which is a beverage, a food, a supplement, a food additive, a quasi-drug, or a medicament.

### Item 13

The composition of any one of the items above, which is in a form of liquid, powder, tablet, or capsule.

### Item 14

The composition of any one of the items above, which is for inducing an exciting feeling.

### Item 15

The composition of any one of the items above, which is for inducing a positive feeling.

### Item 16

The composition of any one of the items above, which is for reducing a decrease in vigor/vitality.

### Item 17

The composition of any one of the items above, which is for alleviating mental stress and/or a feeling of fatigue.

### Item A1

A method for inducing a flow (zone) state in a subject, the method comprising the step of administering a composition comprising γ-aminobutyric acid (GABA) to the subject.

### Item A2

The method of the item above, which is characterized in that the composition is administered before the subject performs an assignment.

### Item A3

The method of any one of the items above, wherein the flow (zone) state is defined at least by distortion of perceived time.

### Item A4

The method of any one of the items above, wherein the flow (zone) state comprises shortening of perceived time.

### Item A5

The method of any one of the items above, wherein the assignment comprises multiple simultaneous tasks.

### Item A6

The method of any one of the items above, wherein the assignment comprises an e-sport, a VDT work, a digital work, and a game.

### Item A7

The method of any one of the items above, wherein the game comprises a TV game, a PC game, and a mobile terminal game.

### Item A8

The method of any one of the items above, wherein the composition is administered from at least about 2 hours before to immediately before the subject performs the assignment.

### Item A9

The method of any one of the items above, wherein a content amount of the γ-aminobutyric acid is about 10 to about 1000 mg.

### Item A10

The method of any one of the items above, wherein an administration amount of the γ-aminobutyric acid is about 10 to about 1000 mg per day.

### Item A11

The method of any one of the items above, which is administered orally.

### Item A12

The method of any one of the items above, wherein the composition is a beverage, a food, a supplement, a food additive, a quasi-drug, or a medicament.

### Item A13

The method of any one of the items above, wherein the composition is in a form of liquid, powder, tablet, or capsule.

### Item A14

The method of any one of the items above, which is for inducing an exciting feeling.

### Item A15

The method of any one of the items above, which is for inducing a positive feeling.

### Item A16

The method of any one of the items above, which is for reducing a decrease in vigor/vitality.

### Item A17

The method of any one of the items above, which is for alleviating mental stress and/or a feeling of fatigue.

### Item B1

A composition for inducing a flow (zone) state, for inducing an exciting feeling, for inducing a positive feeling, for reducing a decrease in vigor/vitality, and/or for alleviating mental stress and/or a feeling of fatigue in a subject, the composition comprising γ-aminobutyric acid (GABA).

### Item B2

The composition f the item above, which is for inducing a flow (zone) state in a subject.

### Item B3

The composition of any one of the items above, which is for inducing an exciting feeling in a subject.

### Item B4

The composition of any one of the items above, which is for inducing a positive feeling in a subject.

### Item B5

The composition of any one of the items above, for reducing a decrease in vigor/vitality in a subject.

### Item B6

The composition of any one of the items above, which is for alleviating mental stress and/or a feeling of fatigue in a subject.

In the present disclosure, in addition to the explicitly shown combinations, the above-mentioned one or more characteristics are intended to be able to be provided in further combinations. Further embodiments and advantages of the present disclosure would be recognized by those skilled in the art by reading and understanding the following detailed explanation as needed.

The characteristics and significant actions/effects of the present disclosure that are not described above would be clear to those skilled in the art by seeing the following embodiment section and drawings of the invention.

### Advantageous Effects of the Invention

According to the present invention, by ingesting a composition comprising GABA, it is possible to induce a flow (zone) state in a subject, thereby enabling efficient performance of work requiring cognitive ability and thinking ability, and providing high utility value in daily life.

In addition, the composition of the present invention can induce a flow (zone) state in a subject, thereby being able to improve productivity and work efficiency in the subject, increase the quality of the task, reduce stress and psychological burden, and/or obtain a sense of fulfillment and a sense of achievement.

### BRIEF DESCRIPTION OF DRAWINGS

FIG. 1 is a schematic view showing the implementation timing of a game test, ingestion of a test beverage, and a VAS questionnaire in one embodiment of the present invention.

FIG. 2 is a graph showing the results of a game test and a VAS questionnaire in one embodiment of the present invention. “GHOST SQUAD™” (SEGA CORPORATION) was used as a game software.

FIG. 3 is a graph showing the results of a game test and a VAS questionnaire in one embodiment of the present invention. “TIME CRISIS 4” (BANDAI NAMCO Games Inc.) was used as a game software.

FIG. 4 is a schematic view showing the implementation timing of a game test and ingestion of a test beverage in one embodiment of the present invention.

FIG. 5 is a graph showing the result of a game test in one embodiment of the present invention.

FIG. 6 is the result showing distortion of perceived time when a placebo, GABA, or caffeine is ingested in one embodiment of the present invention. FIG. 6A is the result of when GABA is ingested, and FIG. 6B is the result of when caffeine is ingested.

FIG. 7 is a schematic view showing a timeline of brainwave data measurement in one embodiment of the present invention.

FIG. 8 is a graph showing the result of a game test in one embodiment of the present invention.

FIG. 9 shows the brainwave measurement result in one embodiment of the present invention.

FIG. 10 is the result of a paired t-test regarding brainwave measurement values in one embodiment of the present invention.

FIG. 11 is a graph showing the difference in SMR waves measured after ingestion of GABA or a placebo relative to the SMR waves at rest in one embodiment of the present invention.

FIG. 12 is a graph showing dart scores in one embodiment of the present invention.

FIG. 13 is the result of measurement of time of a concentration/deep concentration state measured by concentration-measuring glasses (JINS MEME) in one embodiment of the present invention.

FIG. 14 shows the calculation test result in one embodiment of the present invention.

FIG. 15 shows the measurement result of time of a concentration/deep concentration state measured by concentration-measuring glasses (JINS MEME) in one embodiment of the present invention.

### DESCRIPTION OF EMBODIMENTS

Hereinafter, the present disclosure will be explained while showing the best mode. Throughout the entire specification, singular form expressions are to be understood to include the concept of plural forms unless otherwise specified. Accordingly, singular articles (for example, “a”, “an” and “the” in English) are to be understood to include the concept of plural forms unless otherwise specified. In addition, it is to be understood that terms used herein are used in the meaning ordinarily used in the subject field unless otherwise specified. Therefore, unless defined otherwise, all technical and scientific terms used herein have the same meanings as commonly understood by those skilled in the art of the field to which the present disclosure pertains. In the event of any conflict, the present specification (including definitions) shall prevail.

Hereinafter, definitions of terms particularly used herein and/or basic technical contents will be appropriately explained.

As used herein, the term “about” means ±10% of the numerical value that follows.

As used herein, “flow (zone) state” refers to a state in which brain waves in a medium arousal region are dominant, and for example, refers to a state in which SMR (Sensory Motor Rhythm) waves are generated as brain waves. Brain waves in the medium arousal region include, in addition to SMR waves (12 to 15 Hz), high alpha waves (10 to 12 Hz), low beta waves (15 to 18 Hz), and the like. When SMR waves in which relaxation and concentration are balanced are regarded as a peak, a more relaxed state with lower arousal corresponds to high alpha waves, whereas a more concentrated state with higher arousal corresponds to low beta waves. It is known that when SMR waves are generated, a sensation of being in flow or a zone is likely to be experienced (K. Ueda, Y. Imamura and T. Ibaraki, TechRxiv. Preprint, doi:10.36227/techrxiv.14787879.v1 (2021) ). Measurement of brain waves can be performed using a well-known measurement method. For example, the measurement can be performed using various types of brain wave measuring devices such as fMRI, NIRS, invasive-type, MEG, and EEG.

In a flow (zone) state, concentration is enhanced, and when performing a specific action, work, or the like, it is possible to be in a deeper state of concentration than in a normal conscious state. Regarding the flow (zone) state, reference can be made to JeLA Journal Vol. 9/2009.5, “The Application of Flow Theory for Effective E-Learning.” As used herein, flow and zone are synonymous. Whether or not a subject is in a flow (zone) state can be evaluated by distortion of perceived time aside from measurement of brain waves. When a subject performs a specific action, work, or the like, when the time perceived by the subject is shorter than the actual action or work time, it can be said that said subject is in a flow (zone) state.

As used herein, “distortion of perceived time” refers to the concept of temporal experience not coinciding with actual elapsed time, and for example, refers to a case in which, when a subject performs a specific action, work, or the like, the time perceived by the subject is shorter than the actual action or work time. For example, when the perceived time is felt to be less than about 99%, less than about 98%, less than about 97%, less than about 96%, less than about 95%, less than about 94%, less than about 93%, less than about 92%, less than about 91%, less than about 90%, less than about 89%, less than about 88%, less than about 87%, less than about 86%, less than about 85%, less than about 84%, less than about 83%, less than about 82%, less than about 81%, less than about 80%, less than about 75%, less than about 70%, less than about 65%, less than about 60%, less than about 55%, or less than about 50% relative to the actual action or work time, it can be said that distortion of perceived time has occurred.

As used herein, “e-sport” is an abbreviation of “electronic sport” and, in a broad sense, refers to a competition conducted using an electronic device. In a narrow sense, “e-sport” refers to a contest in which a contest using a computer game or a video game is regarded as a sport competition. E-sport competitions include, but are not limited to, sport games, shooting games, simulation games, fighting games, racing games, puzzle games, card games, and RPGs.

As used herein, “VDT work” refers to work using VDTs (Visual Display Terminals) such as a display and a keyboard.

As used herein, “digital work” refers to work mainly utilizing a computer and the Internet. Specific digital work include, for example, communication using a communication tool such as electronic mail or chat, data processing or database management, development of a website or an application, online marketing, and creation of digital content.

### Preferred Embodiments

Preferred embodiments of the present disclosure are described below. The embodiments provided below are provided for better understanding of the present disclosure, and the scope of the present disclosure should not be limited to the following descriptions. Accordingly, it is apparent that those skilled in the art can make appropriate modifications within the scope of the present disclosure with reference to the descriptions herein. In addition, the following embodiments of the present disclosure can be used individually or can be used in combination.

In one aspect of the present invention, a composition for inducing a flow (zone) state in a subject, comprising γ-aminobutyric acid (GABA) is provided.

Since γ-aminobutyric acid (GABA) is an amino acid widely distributed in the natural world such as in vegetables and grain, the origin or the like of GABA is not particularly limited as long as it can be used in drinks or food in one embodiment of the present invention. For example, an extract, a purified product, or the like of a plant containing GABA may be used, or it is possible to carry out preparation from a fermented product obtained by adding glutamic acid decarboxylase, a microorganism having said enzyme such as lactic acid bacteria, or the like to a raw material containing glutamic acid. A product containing GABA or commercially available GABA can also be used for the composition of the present invention in the scope that does not harm the effect caused by the composition of the present invention.

In one embodiment, the composition of the present invention can induce a flow (zone) state in a subject by being ingested by the subject. The flow (zone) state in the subject may be induced when said subject carries out a specific action or work, and said specific action or work may be an assignment imposed on said subject.

In one embodiment, the assignment may be anything that requires at least a certain level of concentration in a subject, and examples thereof include e-sport, VDT work, digital work, games (such as TV games, PC games, and mobile terminal games), and the like. An assignment may be a problem-solving process carried out to achieve a specific objective, and is not limited to those provided in an environment such as a school or a workplace, but also includes those undertaken voluntarily. An assignment is designed to promote personal growth and learning of an individual, and generally can include a specific task or activity for achieving a certain purpose.

In one embodiment, an assignment may be a single task or may be multiple simultaneous tasks. In one embodiment, the multiple simultaneous tasks include a case in which a subject simultaneously performs a plurality of works, actions, assignments, tasks, and/or the like. For example, in the case of VDT work, a single task and multiple simultaneous tasks can include the following works or actions, respectively.

### Single Task

Typing game (repetition of simple data input)
Shooting PC game of simply shooting at a target (only left-clicking a mouse with one hand; a game that does not include weapon switching using a keyboard, switching of the field of view, movement in forward, backward, left, and right directions, movement such as jumping or crouching, or reloading operations of ammunition, or the like)

### Multiple Simultaneous Tasks

Shooting game

While shooting, works such as switching weapons, reloading ammunition, moving forward, backward, left, and right, switching the field of view, and checking a map are performed concurrently.
Racing game

Carrying out acceleration, braking, and determination of left and right directions simultaneously, and the like.

In another embodiment, in the case of tasks other than VDT work, a single task and multiple simultaneous tasks can include the following works or actions, respectively.

### Single Task

Kraepelin test (repetition of simple addition) or the like

### Multiple Simultaneous Tasks

Driving a vehicle: steering operation, operation of turn signals or the like, foot operations, judgments based on observing other vehicles, traffic signals, and people, or the like
Playing the piano: different movements performed by the left and right hands, foot operations, or the like

An e-sport refers to a sport in which computer games or video games (games that are not limited to the devices used, such as PC (Personal Computer), home game console, mobile application, VR, and the like) are carried out as competitions. An e-sport includes, for example, a game that gamifies an actual sport competition, and a game that gamifies a newly created virtual sport competition and is operated by a human player (in the case of a match, an opposing player may be a computer simulation). Furthermore, a virtual sport refers to a sport competition that is modeled after a sport competition actually conducted in the real world, or a newly created virtual sport competition, which is virtually carried out by computer simulation under fixed rules and algorithms. This virtual sport is configured such that a virtual player in data that models an athlete or the like of a sport competition actually conducted in the real world, or a virtual player in data generated by a user under predetermined rules, performs a competition by computer simulation.

In one embodiment, the game genre of e-sports is not particularly limited and may be any game genre. For example, the game may be a fighting game, a sport game, a shooting game, a racing game, a puzzle game, a word game, a card game, a board game, a role-playing game, a simulation game, or the like. In addition, an e-sport may be an individual competition or a team competition. Furthermore, the game may be a competition in which humans compete with each other as players, or a competition in which a human competes with a computer (for example, AI, a robot, or the like).

VDT work refers to work using VDTs (Visual Display Terminals) such as a display and a keyboard. Furthermore, digital work refers to work mainly utilizing a computer and the Internet. Since VDT work and digital work include viewing of a screen for a long period of time, eye fatigue or stress may be caused. The composition of the present invention can also achieve the effect of reducing fatigue or stress in VDT work or digital work by inducing a flow (zone) state in a subject.

In one embodiment, games include those performed using any electronic devices such as TV games, PC games, and mobile terminal games. The game genre is not particularly limited, and may be, for example, a fighting game, a sport game, a shooting game, a racing game, a puzzle game, a word game, a card game, a board game, a role-playing game, a simulation game, or the like. The composition of the present invention can induce a flow (zone) state when a subject plays a game, thereby being able to improve a score or achieve efficient progress in said game.

In one embodiment, the composition of the present invention is ingested before a subject performs an assignment, and may be ingested so that the effect of the composition of the present invention can be obtained. The composition of the present invention can be ingested from at least about 5 hours before up to about 1 minute before, or up to immediately before, the subject performs an assignment. As an upper limit (maximum time) of the ingestion timing, for example, the composition can be ingested at least about 1 hour before, about 2 hours before, about 3 hours before, about 4 hours before, about 5 hours before, about 6 hours before, or about 7 hours before the subject performs an assignment. As a lower limit (minimum time) of the ingestion timing, for example, the composition can be ingested at least about 50 minutes before, about 45 minutes before, about 40 minutes before, about 35 minutes before, about 30 minutes before, about 25 minutes before, about 20 minutes before, about 15 minutes before, about 10 minutes before, about 5 minutes before, about 4 minutes before, about 3 minutes before, about 2 minutes before, about 1 minute before, or immediately before the subject performs an assignment. The ingestion timing can be within a range of numerical values between any of these lower limits and upper limits. By ingesting the composition of the present invention before performing an assignment, it is possible to induce a flow (zone) state when a subject performs said assignment. In one embodiment, how long before performing an assignment the composition of the present invention should be ingested can be appropriately set depending on the type, content, or difficulty of the assignment, the condition or property of the subject, the target depth of the flow (zone) state, or the like.

The composition of the present invention can induce a flow (zone) state in a subject when ingested by the subject. The term “zone” can normally be used in the context of competition or performance. For example, when it is said that athletes “zone” during a match, it means that they are highly focused, action and consciousness are integrated, the boundary between the self and the surroundings becomes ambiguous, and they are in a state in which the highest level of performance can be exerted. By entering the zone, an athlete can exert peak athletic ability without being affected by reduced self-evaluation or worries. The term “flow” can normally be used in the context of creative activities or productive work. For example, when it is said that artists enter a “flow” during a creative activity, it means that they are in a state in which their creative abilities are fully exerted. By entering a flow, an artist can realize the highest level of artistic expression without paying attention to surrounding matters or showing interest in other external interferences. The main difference between zone and flow is that zone usually focuses on competition or performance, whereas flow focuses on creative activities or productive work. However, both concepts represent a state of high concentration and self-containment and indicate a state in which an individual can exert the highest performance. Accordingly, both zone and flow are psychological concepts explaining a state of concentration, and although they can be used differently depending on the category of the subject, they are synonymous herein unless otherwise specified.

A flow state or a zone state can also be expressed as a state in which an ability to execute an assignment in accordance with one's own abilities is improved. In other words, a flow state or a zone state is different from a state of merely having a high level of concentration, and is a state in which engaging in an assignment is enjoyable and one can feel that one's own skills are being enhanced. Even in the case of a high level of concentration, whether or not the state is a flow state or a zone state varies depending on an individual's sensations and experiences, and also varies depending on the content or difficulty of the assignment to be undertaken.

Although not bound by theory, by ingesting the composition of the present invention, it becomes possible to affect brain function via the afferent vagus nerve and to regulate a balance between the sympathetic nerve and the parasympathetic nerve. As a result, it becomes possible to enhance the parasympathetic nerve and it becomes possible to prevent a brain from becoming overactive due to a prolonged state of concentration or tension when engaging in an assignment. Furthermore, it is known that ingestion of GABA can suppress the amount of appearance of 3 waves, and the composition of the present invention can adjust a balance between a waves and 3 waves to create a state in which brain waves in a medium arousal region such as SMR waves are dominant.

In one embodiment, whether or not a subject is in a flow (zone) state can be evaluated at least by distortion of perceived time, and when a specific action, task, or the like is carried out by a subject, when the time felt by the subject is longer or shorter than the actual action or work time, said subject can be defined as being in a flow (zone) state. Distortion of perceived time refers to a state in which actual time does not coincide with perceived time, and for example, when people experience strong emotions such as a state of tension or a pleasant state, time may pass faster or pass slower. Although not bound by theory, this is because the way people recognize the passage of time is affected by factors such as attention and thinking, and when attention and thinking are heightened, people concentrate more attention and process more information, and therefore feel that time passes faster or passes slower. Through causing such distortion of perceived time, the composition of the present invention can induce a flow (zone) state in a subject, and preferably can induce a flow (zone) state when the subject performs a specific assignment.

In one embodiment, when perceived time is shortened, it can be said that a subject is in a flow (zone) state, and shortening of perceived time refers to a case in which, even though the passage of time is the same, time is felt to pass faster.

In one embodiment of the present invention, the content amount of GABA in the composition of the present invention is not particularly limited as long as it is a concentration or weight by which the effect of the composition of the present invention can be obtained, depending on a method of administration or the like. The content amount of GABA in the composition of the present invention is preferably about 10 to about 1000 mg. The lower limit of the content amount can be, for example, about 10 mg or more, about 30 mg or more, about 50 mg or more, about 70 mg or more, about 100 mg or more, about 150 mg or more, about 200 mg or more, about 300 mg or more, about 400 mg or more, about 500 mg or more, about 700 mg or more. The upper limit of the content amount can be, for example, about 1000 mg less, about 800 mg or less, about 700 mg or less, about 600 mg or less, about 500 mg or less, about 400 mg or less, about 300 mg or less, about 200 mg or less, about 100 mg or less. The content amount can be within the range of numerical values between any of these lower limits and upper limits.

In one embodiment of the present invention, the composition of the present invention can comprise, aside from GABA, any additive and any component usable in a composition ingestible in a body, depending on its form. Examples of such additive and component include, but not limited to, vitamins that include vitamin E, vitamin C and the like, minerals, nutritional component, physiologically active component such as flavoring, excipient, binder, emulsifier, tonicity agent (isotonic agent), buffer, solubilizing aid, preservative, stabilizer, antioxidant, colorant, gelling agent, or coating agent used upon formulation, and the like.

In one embodiment of the present invention, the form of the composition of the present invention is not particularly limited as long as GABA is comprised, and for example, the composition of the present invention can be a pharmaceutical composition, a food or drink (including functional food such as food for specified health use, food with nutrient function, and food with function claim, health supplement, health food, supplement, and the like), a food additive, a quasi-drug, or a medicament.

In one embodiment of the present invention, the composition of the present invention can be ingested orally, and when used as an oral agent, its form can be, for example, a tablet agent (including a coated tablet), a capsule agent, a powder agent, a granule agent, a dispersion agent, a liquid agent, a suspension agent, an emulsion agent, a particle-like agent, a powder agent, a pill-like gent, a paste-like agent, a cream-like agent, a caplet-like agent, a gel-like agent, a chewable agent, a stick-like agent, and the like. Furthermore, after being prepared into a form that is easy to mix, such as a powder form or a granule form, use as a raw material for other medicaments is possible.

In one embodiment of the present invention, the composition of the present invention can be used for a non-therapeutic purpose, and can be a non-therapeutic method that induces a flow (zone) state in a subject by ingesting the composition of the present invention. Such a method does not include therapeutic acts or medical acts, and the subject thereof is not particularly limited, which is preferably a healthy person. By having such a healthy person ingest the composition of the present disclosure, a flow (zone) state can be induced. Furthermore, when used as a medicament or a quasi-drug, it is also possible to promote efficacy such as induction of a flow (zone) state.

The composition of the present invention can be used for a therapeutic purpose, and therefore, in another aspect of the present invention, there is provided a method for inducing a flow (zone) state in a subject, the method comprising the step of administering a composition comprising γ-aminobutyric acid (GABA) to the subject.

In one embodiment of the present invention, a daily ingestion amount or a single ingestion amount of GABA comprised in the composition of the present invention can be appropriately set depending on the form of the composition, the ingestion method, the purpose of use, and the age, body weight, symptoms, and the like of the ingestion subject. For example, from the viewpoint of more remarkably exerting the effect of GABA comprised in the composition of the present invention, it is preferable to ingest the composition such that the daily ingestion amount of GABA comprised in the composition of the present invention is about 10 mg/day or more, and it is more preferable to ingest the composition such that the daily ingestion amount is about 25 mg/day or more, about 50 mg/day or more, about 60 mg/day or more, about 80 mg/day or more, about 100 mg/day or more, about 120 mg/day or more, about 150 mg/day or more, about 180 mg/day or more, about 200 mg/day or more, about 300 mg/day or more, about 400 mg/day or more, about 500 mg/day or more, about 800 mg/day or more, or about 1000 mg/day or more. An upper limit of the daily ingestion amount of GABA comprised in the composition of the present invention is not particularly limited as long as the amount is within a range capable of exerting an effect of inducing a flow (zone) state by the composition of the present invention.

In one embodiment of the present invention, an application frequency of the method of the present invention or an ingestion frequency of the composition of the present invention may be once per day or divided into multiple times per day within a desired ingestion amount range, and an ingestion period can also be appropriately set within a range capable of exerting an effect of inducing a flow (zone) state by the composition of the present invention.

In one embodiment, the composition of the present invention can be provided as a composition for inducing an exciting feeling in the subject, independently of induction of a flow (zone) state in the subject. By ingesting the composition of the present invention, an exciting feeling is induced in a subject, thereby being able to improve a score or achieve efficient progress upon undertaking an assignment.

In another embodiment, the composition of the present invention can be provided as a composition for inducing a positive feeling in a subject, independently of induction of a flow (zone) state in the subject. By ingesting the composition of the present invention, a positive feeling is induced in a subject, thereby being able to improve a score or achieve efficient progress upon undertaking an assignment.

In another embodiment, the composition of the present invention can be provided as a composition for reducing a decrease in vigor/vitality in a subject, independently of induction of a flow (zone) state in the subject. By ingesting the composition of the present invention, a decrease in vigor/vitality is reduced in a subject, thereby being able to improve a score or achieve efficient progress upon undertaking an assignment.

In another embodiment, the composition of the present invention can be provided as a composition for alleviating mental stress and/or a feeling of fatigue in a subject, independently of induction of a flow (zone) state in the subject. By ingesting the composition of the present invention, mental stress and/or a feeling of fatigue is alleviated in a subject, thereby being able to improve a score or achieve efficient progress upon undertaking an assignment.

As used herein, the term “or” is used when “at least one or more” of the matters listed in a sentence can be adopted. As used herein, when it is explicitly stated that something is “within a range” between “two values”, the range includes the two values themselves.

The references cited herein such as scientific literature, patents and patent applications, are incorporated herein by reference in their entireties to the same extent as if each reference were individually and specifically indicated to be incorporated by reference.

Accordingly, the present disclosure has been explained above by showing preferred embodiments for ease of understanding. Hereinafter, the present disclosure will be explained based on Examples. However, the above explanation and the following Examples are provided solely for the purpose of illustration, and are not intended to limit the present disclosure. Accordingly, the scope of the present disclosure is not limited to the embodiments or Examples specifically described herein, but is defined only by the Claims.

### EXAMPLES

### Example 1: Game Test 1

In this Example, a change in a game score when GABA was ingested was confirmed.

A total of 10 males and females of age 26 to 33 (first test) and a total of 19 males and females of age 26 to 33 (second test) were used as subjects, and game performance after ingestion of a test beverage was evaluated. The test design was a crossover test.

The test beverages were as follows:
GABA water (PharmaGABA, 200 mg/350 mL)
Red Bull (registered trademark) (arginine 300 mg, caffeine 100 mg/250 ml)
Water (250 ml)

As games, in the first test, a game console: Wii (registered trademark) (Nintendo) and a game software: GHOST SQUAD™ (SEGA) were used, and in the second test, a game console: PlayStation (registered trademark) 3 (Sony Computer Entertainment) and a game software: TIME CRISIS 4 (Bandai Namco Games) were used.

The VAS survey was conducted by administering a questionnaire regarding perceived sensations of items such as “concentration/excitement/positive thinking.” In accordance with the method of Freyd M. et al., the left end of a 100-mm line segment was defined as “not felt at all (=0 mm) ,” and the right end was defined as “felt the most ever (=100 mm) .” The distance between the left end and a position on the line segment at which a subject arbitrarily marked an x was measured and quantified. By taking a difference between a value immediately after ingestion of the test beverage and a value after completion of the test, evaluation was carried out on the perceived sensations felt throughout the test.

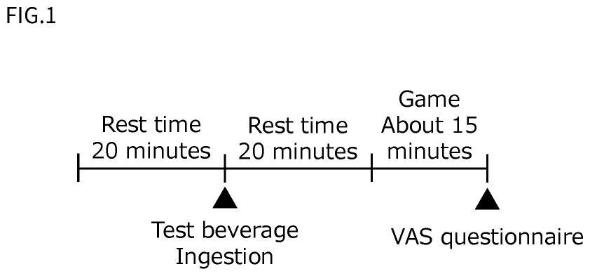

The timing of ingestion of the test beverage, performance of the game, and the VAS questionnaire is as shown in FIG. 1.

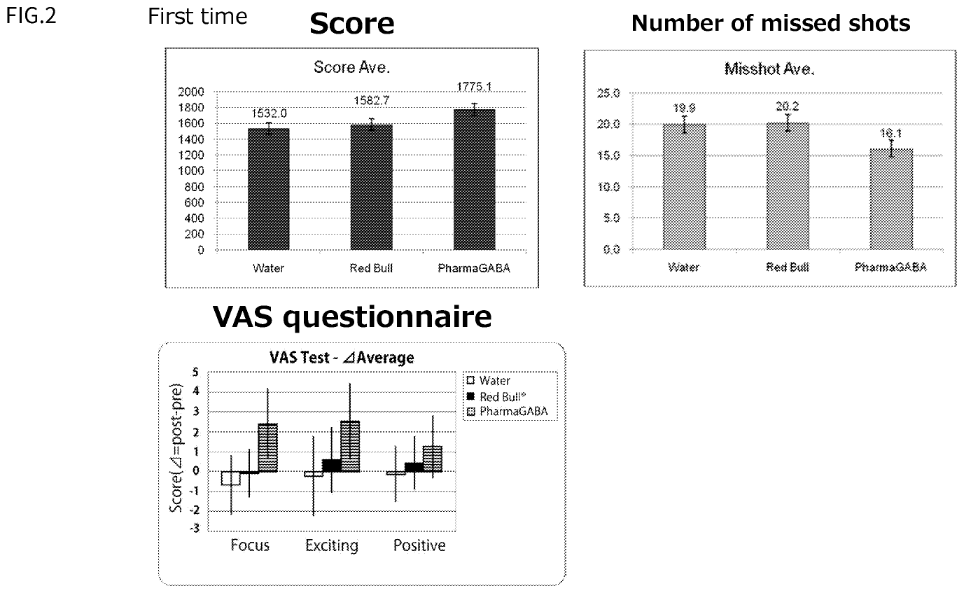

The result of the first test is shown in FIG. 2. As can be seen from FIG. 2, in the first test, compared with a group that ingested water and a group that ingested an energy drink (Red Bull), a group that ingested GABA showed a tendency toward improved game scores, and the number of missed shots was also reduced. Furthermore, from the questionnaire result, it was found that the group that ingested GABA was in an excited state during the game and also had a positive feeling.

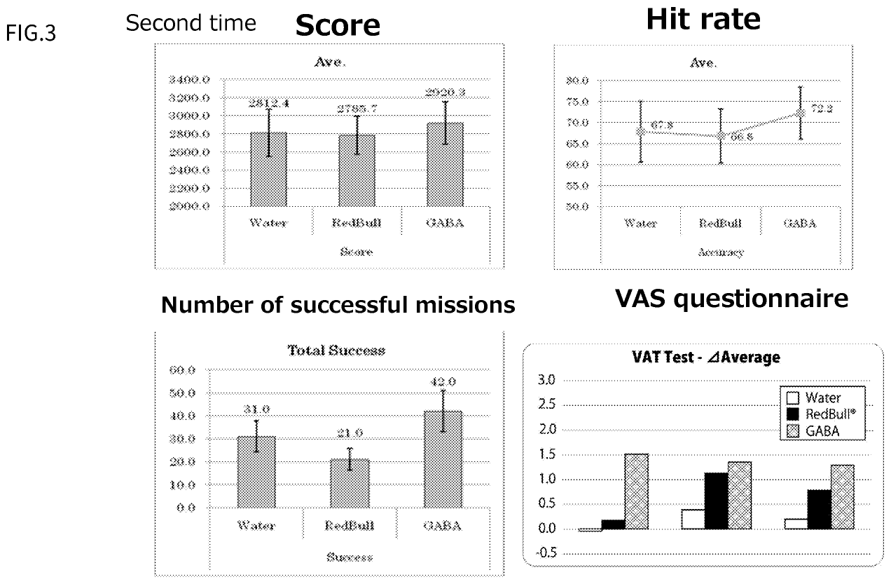

The result of the second test is shown in FIG. 3. As can be seen from FIG. 3, also in the second test, compared with a group that ingested water and a group that ingested an energy drink (Red Bull), a group that ingested GABA showed a tendency toward improved game scores, and the hit rate was improved. Furthermore, in the group that ingested GABA, the number of successful missions during the game was also increased. Furthermore, from the questionnaire result, it was found that the group that ingested GABA was in an excited state during the game and also had a positive feeling.

In both the first test and the second test, ingestion of GABA improved subjective “exciting” scores. At first glance, improvement in the “exciting” scores appears to be inconsistent with a previously reported mechanism in which ingestion of GABA enhances the parasympathetic nerve. However, in known reports, ingestion of GABA in subjects who were given simple calculation problems using a Kraepelin test or the like achieves an effect of reducing stress/feeling of fatigue. On the other hand, in this test, evaluation was performed by imposing a game in which different information is presented every second and different operations are required to be carried out every second. Therefore, in a state of successively processing information as in this test, it is considered that a moderately excited state is important. In other words, it is considered that, as a result of forming a state in which a balance between the sympathetic nerve and the parasympathetic nerve is good by ingestion of GABA, the “exciting” scores were improved. Accordingly, the result of this test does not contradict the existing effect of GABA.

### Example 2: Game Test 2

In this Example, a change in concentration time when GABA was ingested was confirmed.

Seven people who satisfies both “person whose concentration elevates during a game” and “person who has room for elevation in concentration upon ingesting a test food” in screening were used as subjects, and concentration during a game before and after ingestion of a test beverage was evaluated. The test design was a single-blind crossover test, and a washout period was set to one day or more.

The test beverages were as follows:
Placebo beverage: 0.6% sugar water, 250 ml
GABA beverage: 0.6% sugar water containing 100 mg of GABA, 250 ml

As a game, a portable game (Tetris) was used.

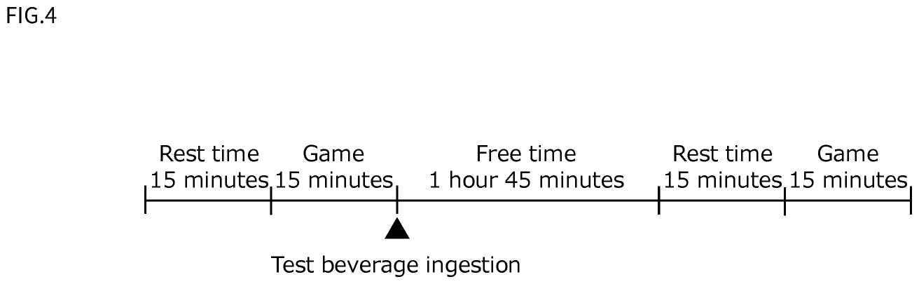

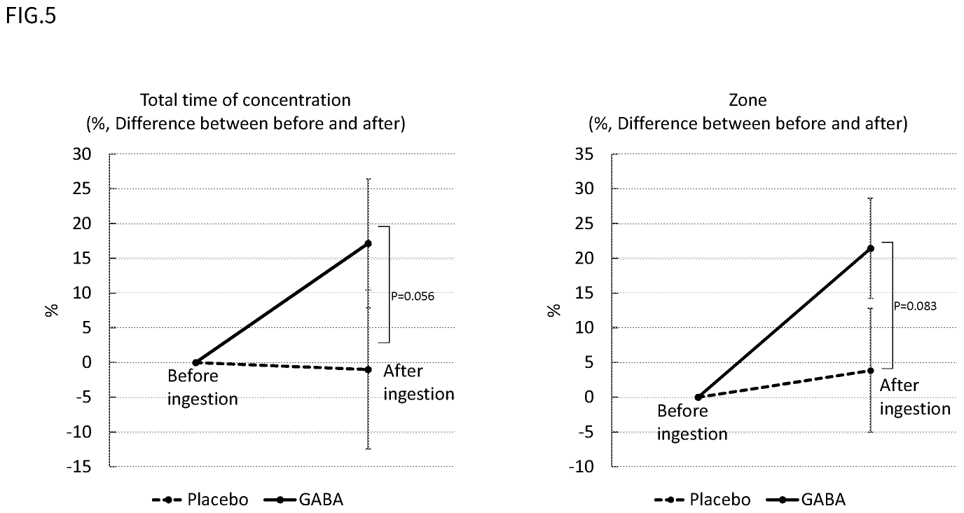

The timing of ingestion of the test beverage and performance of the game is as shown in FIG. 4.

Concentration was measured using JINS MEME ES. With JINS MEME ES, blinking and eye movement are captured from electrooculogram, and concentration and Zone are calculated.

The result is shown in FIG. 5. As can be seen from FIG. 5, when 100 mg of GABA was ingested, the proportion of total concentration time and the proportion of Zone were significantly increased compared with the placebo group. This clarified that concentration and Zone are increased by GABA.

### Example 3: Evaluation of Distortion of Time Interval

In this Example, it was confirmed whether distortion of time interval, which is a characteristic of a flow (zone) state, occurs due to ingestion of GABA.

After ingesting a placebo, GABA, or caffeine, about 15 minutes later, a typing game was carried out for a total of 5 minutes, and time sense evaluation was carried out by a VAS questionnaire. Specifically, the first typing game ended in about 2 minutes (first-half test), and the score was recorded immediately after completion, and the second typing game (second-half test) was started immediately (the interval was about 10 seconds). Thereafter, a questionnaire was conducted regarding how the perceived time felt compared with the actual time (5 minutes).

Furthermore, using concentration-measuring glasses (JINS MEME), time of concentration/deep concentration states was measured. The number of correct inputs (times), the average number of keystrokes (times), and the number of mistypes (times) in the typing game were recorded.

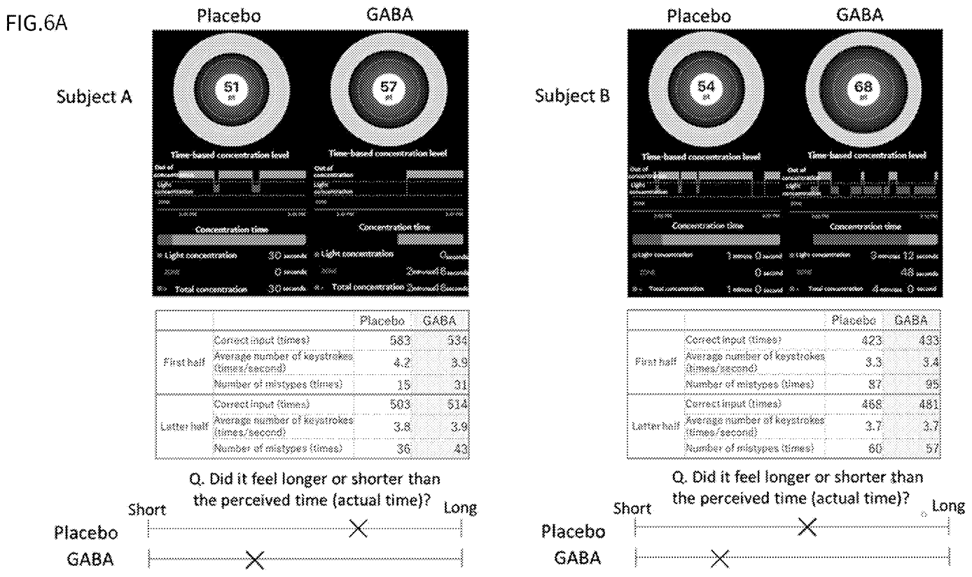

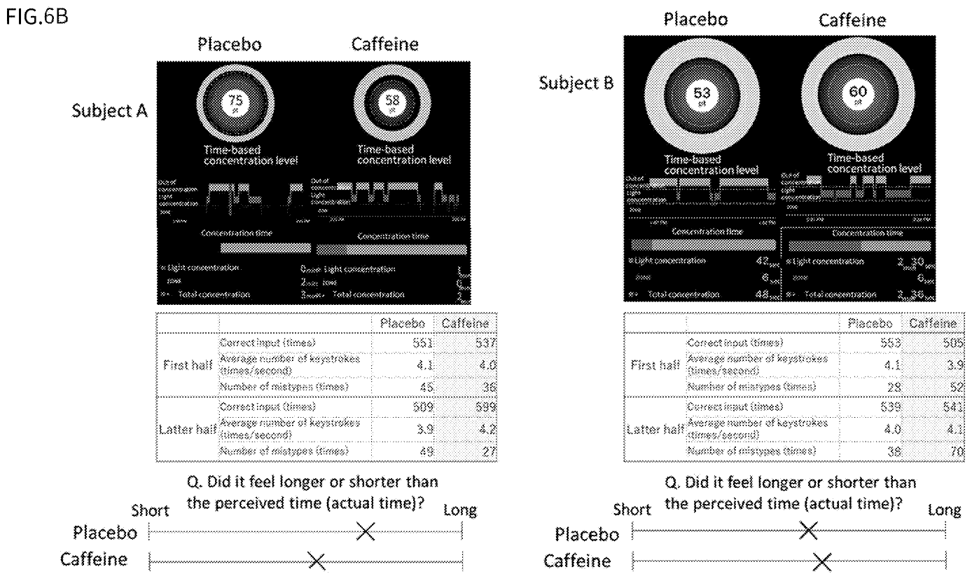

The result is shown in FIGS. 6A and 6B. From this result, it can be seen that concentration time increased compared with the placebo group. Furthermore, it can be seen that, when GABA was ingested, perceived time was felt to be shorter than the actual time. This clarified that GABA causes distortion of perceived time, particularly shortening of perceived time.

### Example 4: Evaluation of the Effect of GABA Ingestion on Brain Wave Data

In this Example, the effect of ingestion of GABA on brain wave data was evaluated, and it was demonstrated that ingestion of GABA induces predominant generation of SMR waves.

### Content of Experiment

### Brain Wave Measurement Experiment

Whether ingestion of GABA affects brain wave data upon performing e-sports was evaluated. The test design was as follows. For brain wave measurement, an analysis algorithm of a brain wave analysis application (Sports Kansei, KANSEI) provided by Little Software Co., Ltd. was used.

### Test Design

Test subjects: 9 people (1 excluded, 8 analyzed)
Device used: simple electroencephalograph
Sample: GABA (200 mg) or placebo (dextrin)+water 30 mL
Measurement items: brain wave, MPG (game assignment) score
Double-blind crossover test (washout period: 1 week)

MPG (Mobalytics Proving Ground) is a game that measures the basic skill of an e-sports player. MPG can be performed using https://pg.mobalytics.gg/.MPG is a game in which three tasks, which are shooting a target (MECHANICS), switching weapons (BG PROCESSING), and checking a map (MAP AWARENESS), are performed simultaneously, and the score for each task and a total score calculated from the three tasks are displayed.

Details of game scores obtained from MPG are as follows. In the following analysis, only the major items indicated by * were used.
(1) Total Score (n)*
(2) Mechanics ability (ability to operate a mouse or keyboard in response to sensory stimulation) (n)*
   Number of target hits (n)
   Accuracy (%)
   Precision (%)
   Ignored targets (n)
   Total lost points (n)
   Center hits (n)
   Center hits among all hits (n)
   Points from center hits (n)
   Middle hits (n)
   Mid-level hits among all hits (n)
   Points from central hits (n)
   Border hits (n)
   Border hits among all hits (n)
   Number of points from border hits (n)
   Average click latency time(s)
   Number of actions per minute (n)
   Lost center points (n)
   Number of target hits (n)
(3) Background processing ability (n)*
   Number of target hits (n)
   Accuracy (%)
   Precision (%)
   Ignored targets (n)
(4) Map cognitive (awareness) ability (n)*
   Score (n)
   Time stuck(s)
   Lost points (n)
   Average time per obstacle(s)

### Test Subjects

The test subjects were nine healthy male university students who were not receiving drug therapy for any disease and did not have symptoms of memory disorder (age: 20.10±1.10 years, height: 178.71±6.96 cm, weight: 60.9±12.90 kg; average value±standard deviation). All test subjects had experience playing League of Legends (23.31 months±11.81), with an average weekly game play time of 3.33 hours±1.25 and an average play frequency of 3.33±1.63 times per week. This test was approved by the ethics committee of Little Software Co., Ltd. (No. 2023-0001, Feb. 28, 2022). The test subjects were informed that they could withdraw from the test at any time, were provided with explanations regarding the purpose, content, method, and the like of the study, and provided written informed consent. The test subject with ID 9 was excluded from the analysis data because he was taking medication for hay fever. Accordingly, valid data were obtained from eight subjects.

### Test Flow

The Test Flow Was As Follows:
(i) 0 to 5 minutes after start of test: explanation of MPG content, pre-implementation, sensitivity setting
(ii) 5 to 15 minutes after start of test: resting state
(iii) 15 to 18 minutes after start of test: brain wave measurement at rest
(iv) 18 to 22 minutes after start of test: POMS 2 implementation (before sample ingestion)
(v) 22 to 23 minutes after start of test: ingestion of sample (GABA or placebo)
(vi) 23 to 55 minutes after start of test: performance of game assignment (32 minutes) and brain wave measurement (three times in total: first half, middle stage, and final stage)
(vii) 55 to 59 minutes after start of test: POMS 2 implementation (after sample ingestion)

In (vi), subjects were asked to perform multiple games with breaks in between.

### Analysis Protocol for Brain Wave Data

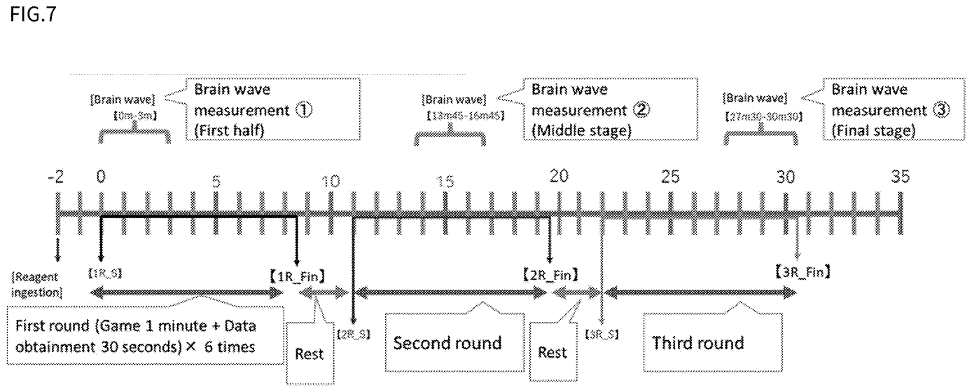

A detailed timeline of brain wave data measurement is shown in FIG. 7.

At each time of brain wave measurement, a waves, B waves, and SMR waves were measured. As SMR waves in this experiment, 12 to 13.25 Hz included in 12 to 15 Hz defined as SMR waves herein was used.

### Results

### Game Scores

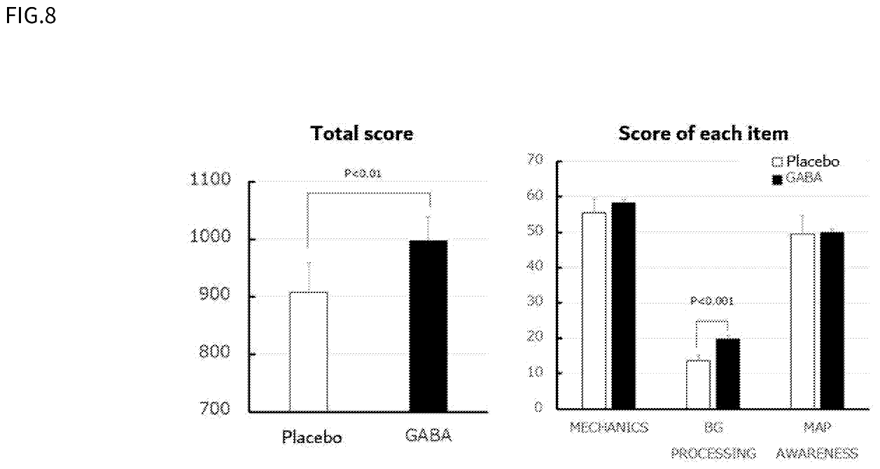

The result of the game scores is shown in FIG. 8. In the GABA ingestion group, the total score was significantly elevated compared with the placebo group.

### Brain Wave Data

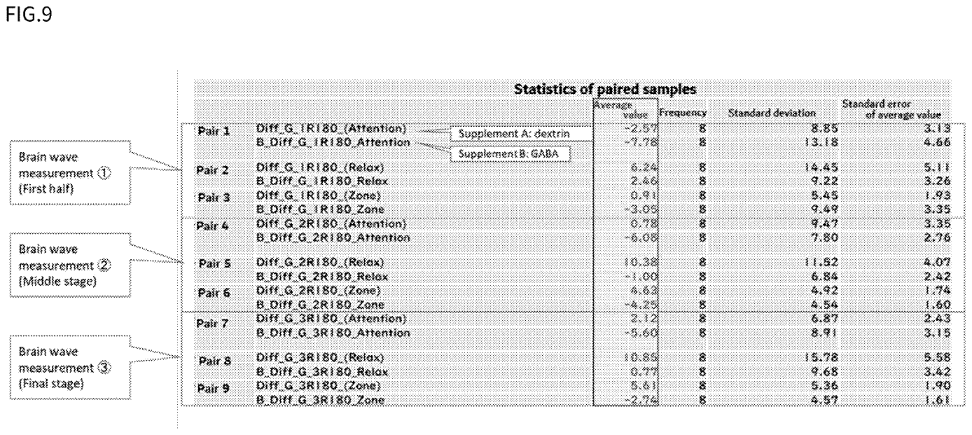

The result of each brain wave measurement (first half, middle stage, and final stage) after ingestion of GABA or placebo (dextrin) is shown in FIG. 9.

The “average value” shown in FIG. 9 indicates, as an amount of change in brain wave measurement values, an average value of “value at rest” minus “value during game play.” In the table, “Attention” indicates β waves, “Relax” indicates a waves, and “Zone” indicates SMR waves.

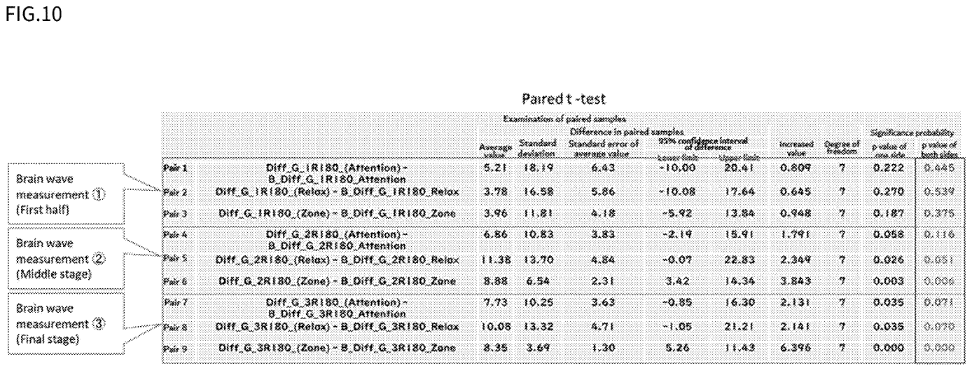

Based on this measurement value, the result of carrying out a paired t-test (n=8) is shown in FIG. 10.

In SMR waves at the final stage of brain wave measurement (“Pair 9” in the table), a significant difference (P<0.001) between GABA ingestion and dextrin ingestion was recognized.

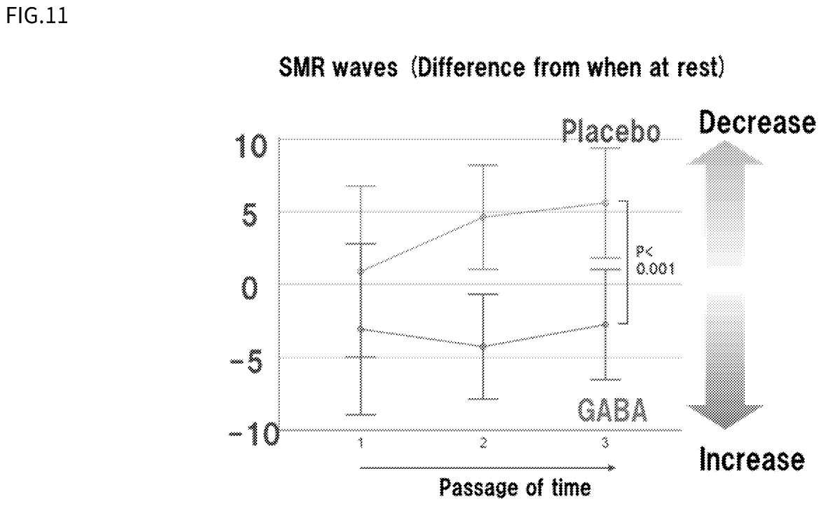

Furthermore, the result of a two-factor analysis of variance (mixed design) (n=8, SMR waves only) is shown in FIG. 11. In FIG. 11, SMR waves after ingestion of GABA or placebo (dextrin) (during game play) are shown as differences relative to those at rest (1: first half, 2: middle stage, 3: final stage). It can be seen that SMR waves increased compared with those at rest due to GABA ingestion.

### Discussion

From the above results, it was demonstrated that GABA ingestion induces predominant generation of SMR waves.

### Example 5: Induction of Flow (Zone) State by GABA Ingestion

In this Example, it is demonstrated that induction of a flow (zone) state by GABA ingestion occurs when performing an e-sport, VDT work, digital work, and/or a game, and that a flow (zone) state is not induced in a test system of monotonous work.

### Content of Experiment

### (1) Experimental Example 1: Darts

Healthy adult men and women drank water in which 200 mg of GABA was dissolved, or placebo (water), and practiced darts. Ingestion of the test food was performed 15 minutes before the start of practice. Using a dartboard, 12 darts were thrown from a distance of 2.37 m. Areas were classified according to distance from the center of the dartboard, and the number of darts stuck in each area was measured. Darts that bounced off the board and did not stick were not counted.

Concentration was measured using JINS MEME. Furthermore, distortion of time sense, which is a characteristic of a flow (zone) state, was evaluated by a VAS questionnaire.

### Results

### Darts Scores

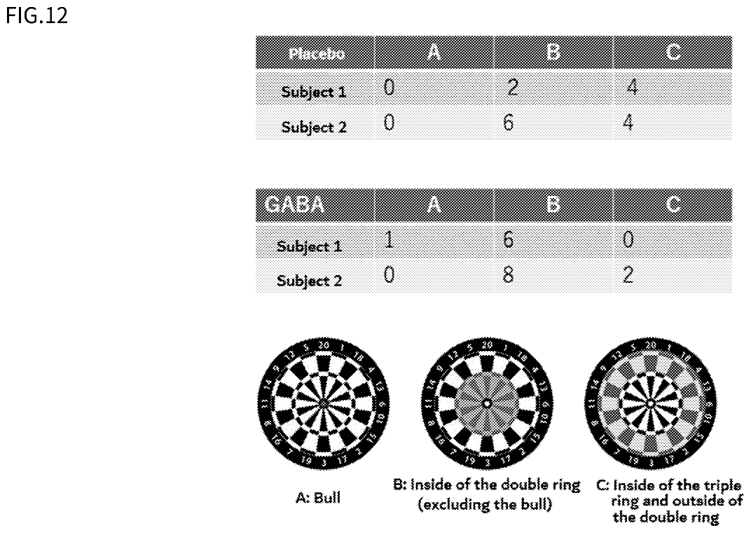

Darts scores are shown in FIG. 12. Counts were made by dividing into A: center (bull), B: inside the double ring excluding the bull, and C: inside the triple ring and outside the double ring. As a result, for both subjects 1 and 2, it can be seen that darts scores were higher upon GABA ingestion than upon placebo ingestion.

### Measurement of Concentration and Evaluation of Distortion of Time Interval

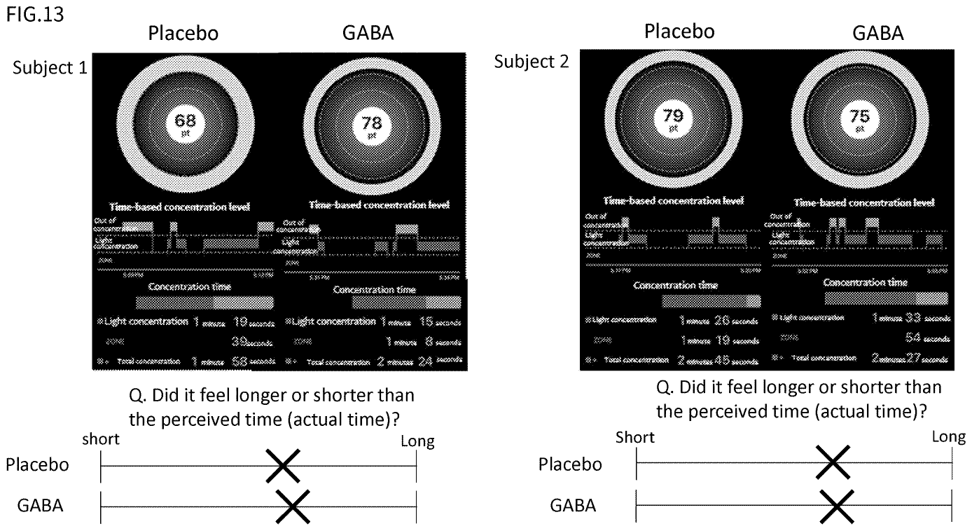

The result of measurement of time of concentration/deep concentration states using concentration-measuring glasses (JINS MEME) is shown in FIG. 13. The result of a VAS questionnaire regarding distortion of time sense is also shown.

From this result, it can be seen that, for both subjects 1 and 2, concentration time increased upon GABA ingestion compared with upon placebo ingestion. On the other hand, no difference was observed in perceived time between upon GABA ingestion and upon placebo ingestion.

### (2) Experimental Example 2: Concentration Evaluation Test Using Calculation Test

Healthy adult men and women drank water in which 200 mg of GABA was dissolved, or placebo (water), and performed a paper-based calculation test. Ingestion of the test food was performed 15 minutes before the start of practice. A calculation test was carried out for 5 minutes, and the number of answers and the correct answer rate were calculated.

### Results

### Test Result

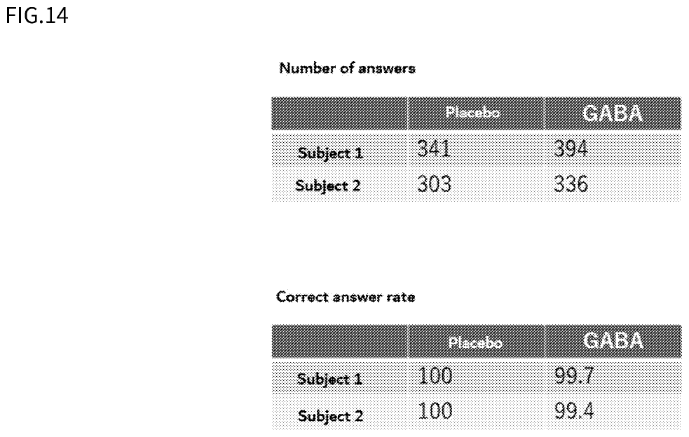

The test result is shown in FIG. 14. For both subjects 1 and 2, it can be seen that the number of answers increased upon GABA ingestion compared with placebo ingestion. Furthermore, no significant difference was observed in the correct answer rate between upon placebo ingestion and upon GABA ingestion.

### Measurement of Concentration and Evaluation of Distortion of Time Interval

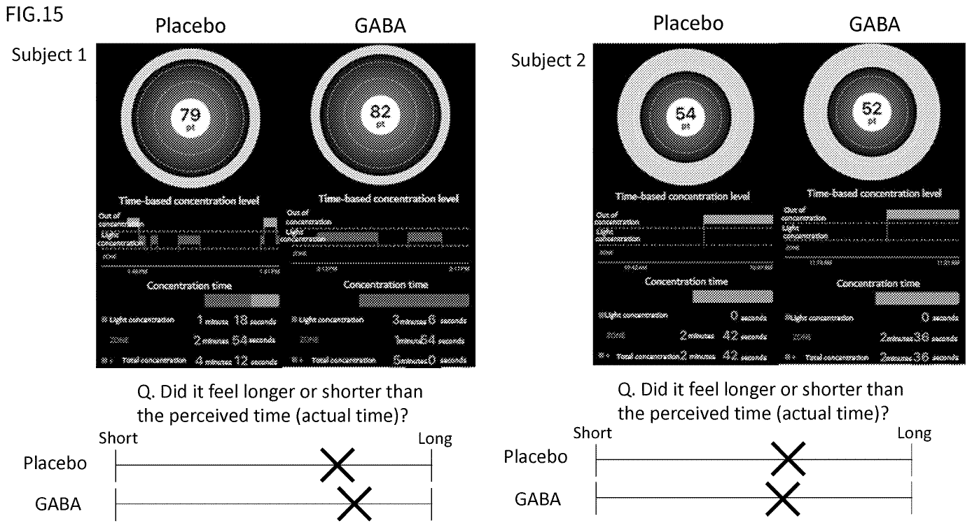

The result of measurement of time of concentration/deep concentration states using concentration-measuring glasses (JINS MEME) is shown in FIG. 15. The result of a VAS questionnaire regarding distortion of time sense is also shown.

From this result, it can be seen that, for both subjects 1 and 2, concentration time increased upon GABA ingestion compared with upon placebo ingestion. On the other hand, no difference was observed in perceived time between upon GABA ingestion and upon placebo ingestion.

### Discussion

From the above result, it was demonstrated that induction of a flow (zone) state by GABA ingestion is not induced in a test system of exercise or monotonous work as described in Cited Reference 1.

### Note

As described above, the present disclosure has been illustrated using preferred embodiments of the present disclosure. However, it is understood that the scope of the present disclosure should be interpreted only by the Claims. It is also understood that the patents, patent applications, and other documents cited herein are incorporated herein by reference as though their contents were specifically described herein. The present application claims priority to Japanese Patent Application No. 2023-141354 filed with the Japan Patent Office on Aug. 31, 2023, and the entire content thereof is incorporated herein by reference as though they constituted the content of the present application.

### INDUSTRIAL APPLICABILITY

According to the present invention, since a flow (zone) state can be induced by ingestion of GABA, the present invention is useful in a field that requires cognitive ability and thinking ability.

1-17. (canceled)

18. A method for inducing a flow (zone) state in a subject, the method comprising the step of administering a composition comprising γ-aminobutyric acid (GABA) to the subject.

19. The method of claim 18, wherein the administration of the composition induces a dominant generation of Sensorimotor Rhythm (SMR) waves of 12 to 15 Hz in the subject, thereby inducing the flow (zone) state.

20. The method of claim 18, wherein the flow (zone) state is induced for performing an e-sport, a VDT work, a digital work, or a game.

21. The method of claim 18, wherein the composition is administered before the subject performs an assignment.

22. The method of claim 18, wherein the flow (zone) state is defined at least by distortion of perceived time.

23. The method of claim 18, wherein the flow (zone) state comprises shortening of perceived time.

24. The method of claim 21, wherein the assignment comprises multiple simultaneous tasks.

25. The method of claim 21, wherein the assignment comprises an e-sport, a VDT work, a digital work, or a game.

26. The method of claim 25, wherein the game comprises a TV game, a PC game, or a mobile terminal game.

27. The method of claim 21, wherein the composition is administered from at least about 2 hours before to immediately before the subject performs the assignment.

28. The method of claim 18, wherein a content amount of the γ-aminobutyric acid is about 10 to about 1000 mg.

29. The method of claim 18, wherein an administration amount of the γ-aminobutyric acid is about 10 to about 1000 mg per day.

30. The method of claim 18, which is administered orally.

31. The method of claim 18, wherein the composition is a beverage, a food, a supplement, a food additive, a quasi-drug, or a medicament.

32. The method of claim 18, wherein the composition is in a form of liquid, powder, tablet, or capsule.

33. The method of claim 18, which is for inducing an exciting feeling.

34. The method of claim 18, which is for inducing a positive feeling.

35. The method of claim 18, which is for reducing a decrease in vigor and/or vitality.

36. The method of claim 18, which is for alleviating mental stress and/or a feeling of fatigue.
